(19) United States
(12) Patent Application Publication
(10) Pub. No.: US 2022/0005581 A1
(43) Pub. Date: Jan. 6, 2022

(54) Systems, Methods, and Devices for Generating and Administering Digital Therapeutic Placebos and Shams

(71) Applicant: Pear Therapeutics, Inc., Boston, MA (US)

(72) Inventors: Michael Brown, Arlington, MA (US); Timothy Campellone, Oakland, CA (US)

(21) Appl. No.: 17/366,278

(22) Filed: Jul. 2, 2021

Related U.S. Application Data
(60) Provisional application No. 63/047,372, filed on Jul. 2, 2020.

(51) Int. Cl. G16H 20/70; G16H 50/70

(52) U.S. Cl. CPC G16H 20/70; G16H 40/20; G16H 50/70

(57) ABSTRACT

A method of treatment comprises administering, via data processing hardware, to a first set of one or more users, a digital therapeutic, wherein the digital therapeutic comprises at least cognitive behavioral therapy content, administering, via the data processing hardware, to a second set of one or more users, at least one of a sham or a placebo digital therapeutic, wherein the at least one sham or placebo digital therapeutic does not comprise any cognitive behavioral therapy content, determining, via the data processing hardware, a first efficacy value associated with the digital therapeutic based on an analysis of engagement of the first set of one or more users with the digital therapeutic, determining, via the data processing hardware, a second efficacy value associated with the at least one sham or placebo digital therapeutic based on analysis of engagement of the second set of one or more users with the sham or placebo digital therapeutic, determining, via the data processing hardware, a third efficacy value associated with the digital therapeutic based on a comparison of the first and second efficacy values, altering, via the data processing hardware, the cognitive behavioral therapy content of the digital therapeutic based on the third efficacy value to provide an updated digital therapeutic, and administering, via the data processing hardware, the updated digital therapeutic to a third set of one or more users.

### CROSS REFERENCE TO RELATED APPLICATIONS

This U.S. patent application claims priority under 35 U.S.C. § 119(e) to U.S. Provisional Application 63/047,372, filed on Jul. 2, 2020. The disclosure of this prior application is considered part of the disclosure of this application and is hereby incorporated by reference in its entirety.

### FIELD

The present disclosure relates to digital therapeutics and, more particularly, to systems, methods, and devices for generating and administering digital therapeutic placebos and shams.

### BACKGROUND

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against the present disclosure.

Drug therapy has played a significant role in the treatment of various medical diseases and disorders. Traditional drug therapy involves the administration of pharmaceuticals and the like. Examples of conventional pharmaceuticals may include small-molecule drugs, which are usually derived from chemical synthesis, and biopharmaceuticals, which may include recombinant proteins, vaccines, blood products used therapeutically gene therapy, monoclonal antibodies, cell therapy, and the like.

While drug therapy has proven to be an effective mechanism for treating certain diseases and disorders, it is not without deficiencies. For example, patients undergoing traditional drug therapy may suffer from undesirable side effects associated with the drugs, administration of traditional drugs may prove challenging, tracking patient adherence to a prescribed drug therapy regimen is often difficult, and traditional drugs are frequently quite costly. Accordingly, digital therapeutics may offer an additional and/or alternative treatment option to traditional drug therapy.

Historically, parameters associated with the performance of a digital therapeutic (e.g., efficacy, safety, patient adherence to a prescription regimen, mitigation of side effects, etc.) for treating a disease or disorder have been evaluated relative to traditional drug therapy for treating that disease or disorder. However, conventional systems and methods for comparing treatment options have proven ineffective for evaluating the performance of a digital therapeutic relative to another digital product.

### SUMMARY

One aspect of the disclosure provides a system for treating a disease or disorder. The system comprises data processing hardware and memory hardware in communication with the data processing hardware, the memory hardware storing instructions that when executed on the data processing hardware cause the data processing hardware to perform operations comprising administering, to a first set of one or more users, a digital therapeutic, wherein the digital therapeutic comprises at least cognitive behavioral therapy content, administering, to a second set of one or more users, at least one of a sham or a placebo digital therapeutic, wherein the at least one sham or placebo digital therapeutic does not comprise any cognitive behavioral therapy content, determining a first efficacy value associated with the digital therapeutic based on an analysis of engagement of the first set of one or more users with the digital therapeutic, determining a second efficacy value associated with the at least one sham or placebo digital therapeutic based on analysis of engagement of the second set of one or more users with the sham or placebo digital therapeutic, determining a third efficacy value associated with the digital therapeutic based on a comparison of the first and second efficacy values, altering the cognitive behavioral therapy content of the digital therapeutic based on the third efficacy value to provide an updated digital therapeutic, and administering the updated digital therapeutic to a third set of one or more users.

Implementations of the disclosure may include one or more of the following optional features. In some implementations, the third set of one or more users may include some or all of either the first or second sets of users.

Administering the sham digital therapeutic may include delivering one or more statements corresponding to general information related to at least one of an emotion, a disease, a disorder, or a condition associated with a user of the second set of one or more users. The one or more statements may be delivered in response to the data processing hardware receiving an indication from a user of the second set of one or more users of an emotion, a disease, a disorder, or a condition that is associated with the user of the second set of one or more users.

Administering the sham digital therapeutic may include generating, for display on a display device, textual content, wherein the textual content is configured to simulate a conversation with a user of the second set of one or more users. The textual content may include at least one of a greeting message, a prompt, a response, or an indication of the termination of the simulated conversation.

Administering the placebo digital therapeutic may include generating, for display on a display device, graphical content, wherein the graphical content comprises a timer.

Administering the placebo digital therapeutic may include generating, for display on a display device, a blank screen.

Administering the sham digital therapeutic may include delivering sham content. Administering the placebo digital therapeutic may include delivering no content.

Another aspect of the disclosure provides a method of treatment comprising administering, via data processing hardware, to a first set of one or more users, a digital therapeutic, wherein the digital therapeutic comprises at least cognitive behavioral therapy content, administering, via the data processing hardware, to a second set of one or more users, at least one of a sham or a placebo digital therapeutic, wherein the at least one sham or placebo digital therapeutic does not comprise any cognitive behavioral therapy content, determining, via the data processing hardware, a first efficacy value associated with the digital therapeutic based on an analysis of engagement of the first set of one or more users with the digital therapeutic, determining, via the data processing hardware, a second efficacy value associated with the at least one sham or placebo digital therapeutic based on analysis of engagement of the second set of one or more users with the sham or placebo digital therapeutic, determining, via the data processing hardware, a third efficacy value associated with the digital therapeutic based on a comparison of the first and second efficacy values, altering, via the data processing hardware, the cognitive behavioral therapy content of the digital therapeutic based on the third efficacy value to provide an updated digital therapeutic, and administering, via the data processing hardware, the updated digital therapeutic to a third set of one or more users. This aspect may include one or more of the following optional features.

In some implementations, the third set of one or more users may include some or all of either the first or second sets of users.

Administering the sham digital therapeutic may include delivering, via the data processing hardware, one or more statements corresponding to general information related to at least one of an emotion, a disease, a disorder, or a condition associated with a user of the second set of one or more users. The one or more statements may be delivered in response to the data processing hardware receiving an indication from a user of the second set of one or more users of an emotion, a disease, a disorder, or a condition that is associated with the user of the second set of one or more users.

Administering the sham digital therapeutic may include generating, via the data processing hardware, for display on a display device, textual content, wherein the textual content is configured to simulate a conversation with a user of the second set of one or more users. The textual content may include at least one of a greeting message, a prompt, a response, or an indication of the termination of the simulated conversation.

Administering the placebo digital therapeutic may include generating, via the data processing hardware, for display on a display device, graphical content, wherein the graphical content comprises a timer.

Administering the placebo digital therapeutic may include generating, via the data processing hardware, for display on a display device, a blank screen.

Administering the sham digital therapeutic may include delivering, via the data processing hardware, sham content. Administering the placebo digital therapeutic may include delivering, via the data processing hardware, no content.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 is a schematic view of an example system including a digital therapeutic placebo/sham application in accordance with principles of the present disclosure;

FIG. 2 is a flowchart of an example pre-study operation of the digital therapeutic placebo/sham application of FIG. 1;

FIG. 3A is a flowchart of an example startup operation of the digital therapeutic placebo/sham application of FIG. 1;

FIG. 3B is a flowchart of an example treatment group operation of the digital therapeutic placebo/sham application of FIG. 1;

FIG. 3C is a flowchart of an example placebo/sham group operation of the digital therapeutic placebo application of FIG. 1;

FIG. 4 is a flowchart of an example study operation of the digital therapeutic placebo/sham application of FIG. 1;

FIG. 5 is a flowchart of an example treatment operation of an authentic digital therapeutic application in accordance with principles of the present disclosure;

FIG. 6A illustrates a user device having a first graphical user interface of the digital therapeutic placebo/sham application of FIG. 1;

FIG. 6B illustrates a user device having a second graphical user interface of the digital therapeutic placebo/sham application of FIG. 1;

FIG. 7A illustrates a user device having a first graphical user interface of an exemplary authentic digital therapeutic application;

FIG. 7B illustrates a user device having a second graphical user interface of the exemplary authentic digital therapeutic application of FIG. 7A;

FIG. 8 is a flowchart of a method in accordance with principles of the present disclosure; and

FIG. 9 is a schematic view of an example computing device that may be used to implement the systems and methods described herein.

Like reference symbols in the various drawings indicate like elements.

### DETAILED DESCRIPTION

Example embodiments are provided so that this disclosure will be thorough, and will fully convey the scope to those who are skilled in the art. Numerous specific details are set forth, such as examples of specific systems, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

As shown and described more fully below, the instant disclosure includes systems, methods, and devices for treating various diseases and disorders. More specifically, the instant disclosure includes systems, methods, and devices for generating and administering a digital therapeutic placebo and/or a digital therapeutic sham. The systems described herein may include computing components connected over one or more wired or wireless networks for storing digital therapeutic placebos/shams, transmitting digital therapeutic placebos/shams to users' computing devices (e.g., computing devices associated with participants in a study or trial designed to evaluate the performance of a digital therapeutic for treating a given disease or disorder), and evaluating the performance of a digital therapeutic by comparing user-interaction with the digital therapeutic with user-interaction with a corresponding digital therapeutic placebo/sham. The methods described herein may include methods for administering a digital therapeutic placebo/sham to a user, methods for evaluating the performance of a digital therapeutic by comparing user-interaction with the digital therapeutic with user-interaction with a corresponding digital therapeutic placebo/sham, and methods for altering the digital therapeutic and/or the digital therapeutic placebo/sham based on the evaluation of performance. The devices described herein may include computing devices (e.g., server computer(s), smartphone(s), tablet(s), desktop computer(s), laptop computer(s), or any combination thereof) for storing, transmitting, altering, and/or executing a patient-facing placebo/sham application. The patient-facing placebo/sham application may constitute a mobile application, a web-based application, or any combination thereof.

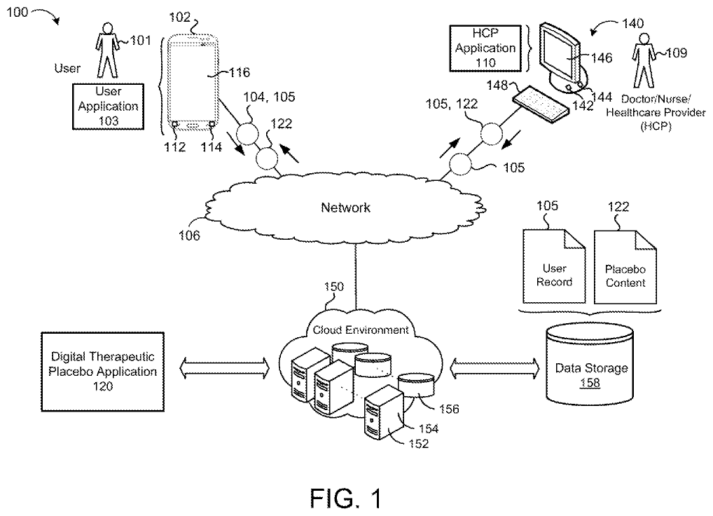

Digital therapeutics may provide therapeutic content to patients in the form of textual and/or audio/visual content delivered through a computer application executable on a computing device. To evaluate the performance of a digital therapeutic (e.g., across performance parameters such as efficacy, safety, patient adherence to a prescribed regimen, or any other performance parameters for medical treatment known in the art) relative to a non-drug counterpart (e.g., as compared to a digital counterpart), a digital therapeutic placebo/sham may be implemented. According to one example, a first group of users (e.g., participants in a study or trial designed to evaluate the performance of a digital therapeutic for treating a given disease or disorder) may be administered a digital therapeutic designed to treat a particular disease or disorder. For example, the digital therapeutic may be provided from a host server or the like to computing devices associated with the first group of users over a networked system, such as the networked system shown in FIG. 1. This first group of users may be referred to as a “Treatment Group.”

In addition, a second group of users (e.g., participants in a study or trial designed to evaluate the performance of a digital therapeutic for treating a given disease or disorder) may be administered a digital therapeutic placebo/sham that is not designed to treat the particular disease or disorder. For example, the digital therapeutic placebo/sham may be provided from a host server or the like to computing devices associated with the second group of users over a networked system, such as the networked system shown in FIG. 1. This second group of users may be referred to as a “Placebo/Sham,” or “Control” Group.

According to exemplary implementations described herein, the digital therapeutic placebos may administer content that, while visually or audibly resembles therapeutic content (e.g., cognitive behavioral therapeutic content), does not actually administer any therapeutic content capable of treating the disease or disorder that the corresponding actual or authentic digital therapeutic is designed to treat. In one example implementation, a digital therapeutic placebo may administer substantially no content (e.g., no textual content) whatsoever, and instead may generate a blank screen. In another example implementation, the digital therapeutics placebo may administer a timer or the like, whereby the timer or the like does not deliver any therapeutic content capable of treating the disease or disorder that the corresponding actual or authentic digital therapeutic is designed to treat. In another example implementation, the second group of users may be placed on a waitlist where they do not receive the digital therapeutic, and, instead, the waitlist group waits to receive the digital therapeutic and may continue to receive a standard of care (i.e., absent the use of a digital therapeutic) for the particular disease or disorder they are facing. At some point in the future, the waitlist group may eventually receive the digital therapeutic.

According to another example implementation, a digital therapeutic sham may administer “sham” content, such as generic or psycho-educational content that is specifically designed to be devoid of therapeutic content (e.g., cognitive behavioral therapeutic content) for treating the disease or disorder that the corresponding actual digital therapeutic is designed to treat. Additional, non-limiting, examples of sham content may include textual content that has not been demonstrated to treat the disease or disorder that the corresponding actual digital therapeutic is designed to treat (e.g., the textual content shown in Table 1), visual content that has not been demonstrated to treat the disease or disorder that the corresponding actual digital therapeutic is designed to treat, audio content that has not been demonstrated to treat the disease or disorder that the corresponding actual digital therapeutic is designed to treat, or any combination thereof.

According to one example, a digital therapeutic may be configured to treat depressive symptoms associated with one or more diseases or disorders (e.g., multiple sclerosis). According to this example, the sham content generated by the digital therapeutic sham may include textual, audio, and/or visual content directed to an emotional state that a user is experiencing (e.g., based on input information provided by the user through the digital therapeutic sham application). Continuing with this example, in a situation in which a user indicates that they are experiencing a lonely emotional state, the digital therapeutic sham may generate display data for display on the user's computing device conveying a message such as “Isolation compromises immunity, increases the production of stress hormones, is harmful to sleep, and impacts cognitive abilities,” which message has been demonstrated not to have any therapeutic effect on mitigating or eliminating depressive symptoms associated with one or more diseases or disorders. Additional exemplary sham content is illustrated in Table 1. It should be understood that different and/or additional sham content is contemplated.

In some implementations, the digital therapeutic placebo/sham delivered to the Placebo/Sham/Control Group may include functionality and/or content that is similar to what is included in the corresponding actual digital therapeutic provided to the Treatment Group. For example, the digital therapeutic placebo/sham may allow the Placebo/Sham/Control Group to: (i) access and interact with content through the user's computing device (e.g., the user's mobile device) via a conversational graphical user interface included as part of the digital therapeutic placebo/sham application; (ii) log into the digital therapeutic placebo/sham application via a login interface (e.g., by the user inputting a unique ID and access code); and/or (iii) receive local notifications on the user's computing device that, when selected by the user, initiate the digital therapeutic placebo/sham application on the user's computing device. In some implementations, repeating similar conversation scenarios may result in delivering the same copy of the sham (e.g., psycho-educational) content to the user in the Placebo/Sham/Control Group as the previous scenario; that is, the user may not be delivered different sham (e.g., psycho-educational) content across subsequent sessions with the digital therapeutic placebo/sham application.

In some implementations, the digital placebo/sham application may deliver three daily notifications prompting the user to open the digital placebo/sham application, and then display a prescription timer (e.g., a timer clock countdown until the next prescription dose) for the remaining duration of app availability (e.g., for the duration of a prescription from a treating physical). The digital placebo/sham application may account for nonspecific effects of engagement with a smartphone. The digital placebo/sham application may not deliver any active coping skills (e.g., ingredients of a psychosocial intervention), however, it may appear visually similar to the actual digital therapeutic.

The systems, methods, and devices described herein may be used to evaluate the effectiveness of actual digital therapeutics designed to treat diseases and disorders including, but not limited to, substance use disorder, opioid use disorder, schizophrenia, post-traumatic stress disorder, general anxiety disorder, insomnia, traumatic brain injury, attention-deficit/hyperactivity disorder, multiple sclerosis, and major depressive disorder, among others.

Referring to FIG. 1, in some implementations, a digital therapeutic placebo/sham system 100 provides a user 101 access to a digital therapeutic placebo/sham application 120 assigned to the user 101, and monitors events associated with the user's 101 interaction with the digital therapeutic placebo/sham application 120. Although the digital therapeutic placebo/sham application 120 is described herein as being a “placebo/sham” digital therapeutic, it is understood that, according to some implementations, the digital therapeutic placebo/sham application 120 may function similar to an authentic digital therapeutic, e.g., a prescription digital therapeutic, such that the user 101 is unaware whether they are interacting with an authentic digital therapeutic or the digital therapeutic placebo/sham. The person using or being administered the digital therapeutic placebo/sham may be referred to as a “user.” A “user” may include a patient, a user in the Placebo/Sham or Control Group, or any other person using or being administered the digital therapeutic placebo/sham application 120.

As used herein, a digital therapy may also be referred to as a digital-therapeutic configured to deliver evidence-based psychosocial intervention techniques for treating a patient with a particular disease or disorder, as well as symptoms and/or behaviors associated with the particular disease or disorder. In some implementations, an authorized healthcare provider (HCP) 109 (e.g., a doctor, nurse, etc.) may supervise the user 101 and may assign the digital therapeutic placebo/sham application 120 to the user 101. The HCP 109 may include a physician, nurse, clinician, or other qualified health professionals. In other implementations, the user 101 may be assigned the digital therapeutic placebo/sham application 120 by any suitable person, such as someone involved in a clinical trial.

In some examples, the system 100 includes a network 106, a user device 102, an optional HCP system 140, and the digital therapeutic placebo/sham application 120. The network 106 provides access to cloud computing resources 150 (e.g., distributed system) that execute the placebo/sham application 120 to provide for the performance of services on remote devices. The network 106 may include any type of network that allows sending and receiving communication signals, such as a wireless telecommunication network, a cellular telephone network, a time division multiple access (TDMA) network, a code division multiple access (CDMA) network, Global system for mobile communications (GSM), a third generation (3G) network, fourth generation (4G) network, fifth generation (5G) network, a satellite communications network, and other communication networks. The network 106 may include one or more of a Wide Area Network (WAN), a Local Area Network (LAN), and a Personal Area Network (PAN). In some examples, the network 106 includes a combination of data networks, telecommunication networks, and a combination of data and telecommunication networks. The user device 102, the optional HCP system 140, and the placebo/sham application 120 communicate with each other by sending and receiving signals (wired or wireless) via the network 106. In some examples, the network 106 provides access to cloud computing resources, which may be elastic/on-demand computing and/or storage resources 156 available over the network 106. The term “cloud” services generally refers to a service performed not locally on a user's device, but rather delivered from one or more remote devices accessible via one or more networks 106.

The user device 102 may include, but is not limited to, a portable electronic device (e.g., smartphone, cellular phone, personal digital assistant, personal computer, or wireless tablet device), a desktop computer, or any other electronic device capable of sending and receiving information via the network 106. The user device 102 includes data processing hardware 112 (a computing device that executes instructions), memory hardware 114, and a display 116 in communication with the data processing hardware 112. In some examples, the user device 102 includes a keyboard, mouse, microphones, and/or a camera for allowing the user 101 to input data. In addition to or in lieu of the display 116, the user device 102 may include one or more speakers to output audio data to the user 101. For instance, audible alerts may be output by the speaker to notify the user 101 about some time sensitive event associated with the placebo/sham application 120. In some implementations, the user device 102 executes a user application 103 (or accesses a web-based patient application) for establishing a connection with and accessing the placebo/sham application 120. For instance, the user 101 may have access to the user application 103 for a duration (e.g., 3 months) of the placebo/sham application 120 assigned to the user 101. Here, the user device 102 may launch the user application 103 by initially providing an access code 104 when the placebo/sham application 120 is assigned to the user 101 that allows the user 101 to access content associated with the placebo/sham application 120. The user application 103, when executing on the data processing hardware 112 of the user device 102, is configured to display a variety of graphical user interfaces (GUIs) on the display 116 of the user device 102 that, among other things, allow the user 101 to interact with the placebo/sham application 120.

The storage resources 156 may provide data storage 158 for storing a user record 105 and placebo/sham content 122. The user record 105 may be encrypted while stored on the data storage 158 so that any information identifying user 101 is anonymized, but may later be decrypted when the user 101 or supervising HCP 109, or clinical trial worker, requests the user record 105 (assuming the requester is authorized/authenticated to access the user record 105). All data transmitted over the network 106 between the user device 102 and the cloud computing system 150 may be encrypted and sent over secure communication channels.

The optional HCP system 140 may be located at a clinic, doctor's office, or facility administered by the HCP 109 and includes data processing hardware 142, memory hardware 144, and a display 146. The memory hardware 144 and the display 146 are in communication with the data processing hardware 142. For instance, the data processing hardware 142 may reside on a desktop computer or portable electronic device for allowing the HCP 109 to input and retrieve data to and from the placebo/sham application 120. In some examples, the HCP 109 may initially onboard some or all of the user record 105 and/or the placebo/sham content 122. In other examples, the user 101 may onboard some or all of the user record 105 and the placebo/sham content 122 may be created by clinical trial workers or any other suitable person. The HCP system 140 includes a keyboard 148, mouse, microphones, speakers and/or a camera. In some implementations, the HCP system 140 (i.e., via the data processing hardware 142) executes a HCP application 110 (or accesses a web-based patient application) for establishing a connection with the placebo/sham application 120 to input and retrieve data therefrom.

The cloud computing resources 150 may be a distributed system (e.g., remote environment) having scalable/elastic resources 152. The resources 152 include computing resources 154 (e.g., data processing hardware) and/or the storage resources 156 (e.g., memory hardware). The cloud computing resources 150 execute the placebo/sham application 120 for facilitating communications with the user device 102 and the HCP system 140 and storing data on the storage resources 156 within the data storage 158. In some examples, the data storage 158 resides on a standalone computing device. The placebo/sham application 120 may provide the user 101 with the user application 103 (e.g., a mobile application, a web-site application, or a downloadable program that includes a set of instructions) executable on the data processing hardware 112 and accessible through the network 106 via the user device 102 when the user 101 provides a valid access code 104. Similarly, the placebo/sham application 120 may provide the HCP 109 with the HCP application 110 (e.g., a mobile application, a web-site application, or a downloadable program that includes a set of instructions) executable on the data processing hardware 142 and accessible through the network 106 via the HCP system 140.

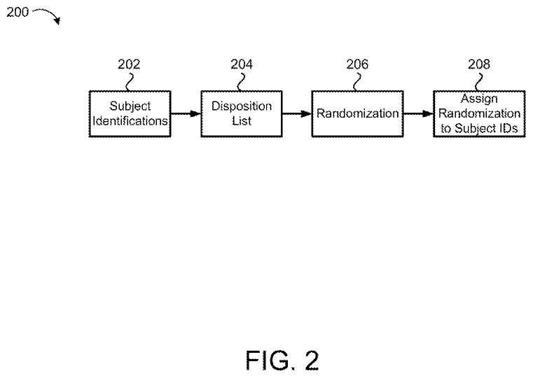

FIG. 2 is a flowchart showing a pre-study operation 200 according to one example implementation of the present disclosure. During the pre-study operation 200, the system 100 may identify the subjects or patients of the study at node 202. The system 100 may then develop a disposition list at node 204 to be used in the randomization of the subjects or patients at node 206. The identification and randomization of the patients may be performed by a third-party, the party conducting the study, any other suitable party, or any suitable combination of the foregoing. During the randomization process, each of the patients may be assigned a unique identification name or number at node 208.

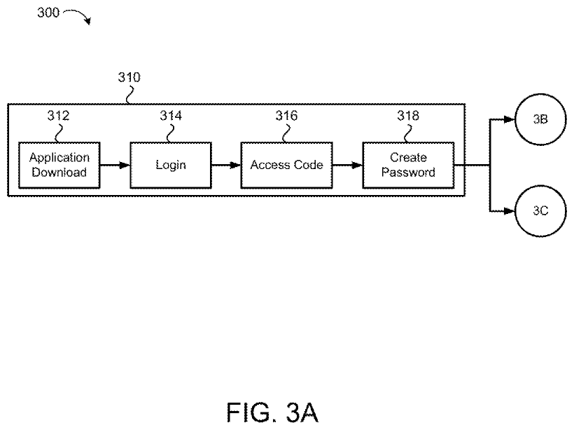

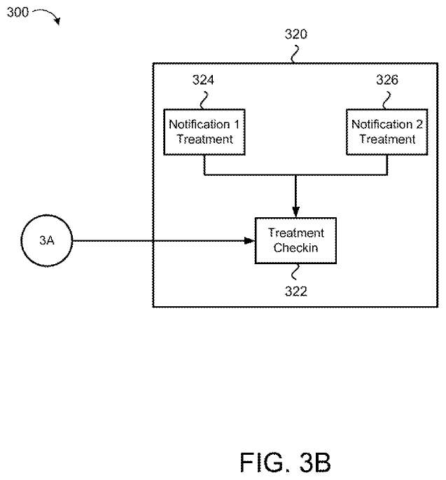

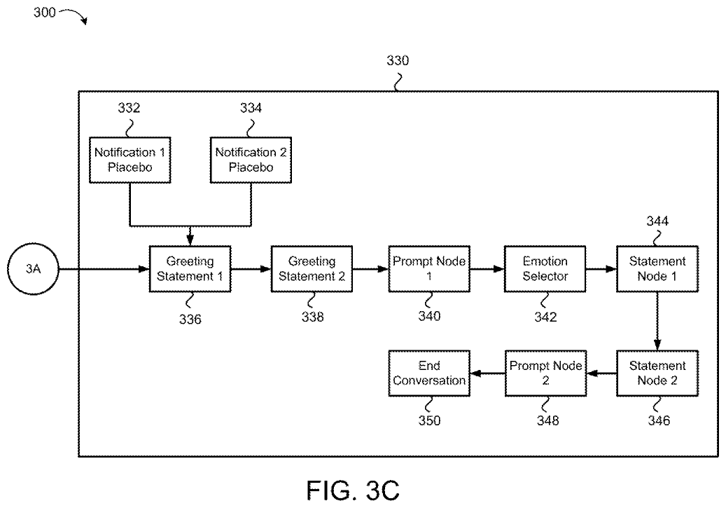

FIGS. 3A-3C are flowcharts showing an application operation 300 according to one example implementation of the present disclosure. Referring to FIG. 3A, a startup operation 310 is generally shown. During the startup operation 310, the system 100 may enable the patients to download the user application 103 at node 312, e.g., via an app store. During a login process at node 314, the patient may provide the access code 104 at node 316, which may enable the patient to complete the login process, e.g., create a password at node 318, create a username, etc. During the startup operation 310, the patients of each group (i.e., the treatment group and the placebo/sham group) may experience a substantially similar process such that the patients do not know which group they are in. For example, the patients in the placebo/sham group may not know whether they are in the placebo/sham group or the treatment group.

Referring to FIG. 3B, a treatment group operation 320 is generally shown. During the treatment group operation 320, patients of the study group may experience a routine of receiving treatment—shown as a first notification treatment 324 and a second notification treatment 326—and checking in with the HCP 109 via interaction with the user application 103, in person meetings, or any other suitable means at node 322.

Referring to FIG. 3C, a placebo/sham group operation 330 is generally shown. During the placebo/sham group operation 330, the digital therapeutic placebo/sham application 120 may present to the patients of the placebo/sham group, e.g., via interaction with the user application 103, textual content that has not been demonstrated to treat the disease or disorder that the corresponding actual digital therapeutic is designed to treat (e.g., the textual content shown in Table 1), visual content that has not been demonstrated to treat the disease or disorder that the corresponding actual digital therapeutic is designed to treat, audio content that has not been demonstrated to treat the disease or disorder that the corresponding actual digital therapeutic is designed to treat, or any combination thereof. Such content may be part of a sham digital therapeutic.

In other implementations, the digital therapeutic placebo/sham application 120 may administer a placebo digital therapeutic, which includes the same startup operation 310 as the sham digital therapeutic and an authentic digital therapeutic, but, after the startup operation 310, the placebo digital therapeutic presents substantially no content. As one example, the placebo digital therapeutic may generate and display on the display 116 a blank screen. As another example, the placebo digital therapeutic may generate and display on the display 116 a timer, e.g., a countdown timer.

In some implementations, the digital therapeutic placebo/sham application 120 may present to the patient (e.g., by displaying on the display 116) a first placebo notification 332 and a second placebo notification 334. The first and second placebo notifications 332, 334 may be a push notification or other similar notifications to remind the patient to use the user application 103. Once the patient opens the digital therapeutic placebo/sham application 120 (e.g., through interaction with the user application 103), the digital therapeutic placebo/sham application 120 may simulate a conversation with the user 101. For example, the digital therapeutic placebo/sham application 120 may present to the patient a first greeting statement at node 336. After, the digital therapeutic placebo/sham application 120 may present to the patient a second greeting statement at node 338. The first and second greeting statements 336, 338 may include textual statements such as “Hello,” and “Welcome back to the app,” as shown in Table 1 below. At node 340, the digital therapeutic placebo/sham application 120 may present to the patient a first prompt, such as “How are you feeling?” At node 342, the digital therapeutic placebo/sham application 120 may receive from the patient, e.g., via the patient's interaction with the user application 103, an indication of the user's emotion. In some implementations, the digital therapeutic placebo/sham application 120 may present to the patient a plurality of selectable emotions, including, but not limited to, lonely, anger, afraid, grief, stressed, depressed, frustrated, shame, happy, optimistic, proud, motivated, compassionate, loving, calm, okay, etc., as shown in Table 1. In other implementations, the patient may enter their emotion through a freetext entry field, speak their emotion, or submit their emotion in any suitable manner.

At nodes 344 and 346, the digital therapeutic placebo/sham application 120 may present to the patient a first statement and a second statement, respectively, based on the selected emotion. As shown in Table 1, the first and second statements 344, 346 may include general facts or relatively generic information regarding the selected emotion, the specific disease or disorder that is being treated, other data, etc. For example, if the patient selects “Anger” as their emotion, the digital therapeutic placebo/sham application 120 may display on the display 116 “The overall prevalence of inappropriate, intense, or poorly controlled anger in the U.S. population is 7.8%,” corresponding to “SHAM ANGER 1” in Table 1. As shown in Table 1, the digital therapeutic placebo/sham application 120 may present to the patient multiple statements at nodes 344 and 346 if the patient selects the same emotion multiple times. For example, if the patient selects “Anger” as their emotion after having already selected this emotion, the digital therapeutic placebo/sham application 120 may display on the display 116 “Increased blood pressure and other physical changes associated with anger make it difficult to think straight and can harm your physical and mental health,” corresponding to “SHAM ANGER 2” in Table 1. If the patient were to select “Anger” as their emotion again, in some implementations, the digital therapeutic placebo/sham application 120 would present the statement corresponding to “SHAM ANGER 1.” In other implementations, the digital therapeutic placebo/sham application 120 may include several different statements to present to the patient.

At node 348, the digital therapeutic placebo/sham application 120 may present to the patient a second prompt such as “Are you feeling better?” At node 350, the digital therapeutic placebo/sham application 120 may terminate the conversation or interaction with the patient, e.g., by displaying on the display 116 “End” or “Done.”

TABLE 1

| Identifier | Statement |
|---|---|
| SHAM 1 | Hello. |
| SHAM 2 | Welcome back to the app. |
| SHAM LONELY 1 | Cigna’s 2018 Loneliness Index found that 46% of |
| | Americans report feeling lonely or left out sometimes. |
| SHAM LONELY 2 | Isolation compromises immunity, increases |
| | the production of stress hormones, is harmful |
| | to sleep, and impacts cognitive abilities. |
| SHAM ANGER 1 | The overall prevalence of inappropriate, |
| | intense, or poorly controlled anger in the U.S. |
| | population is 7.8%. |
| SHAM ANGER 2 | Increased blood pressure and other physical |
| | changes associated with anger make it difficult |
| | to think straight and can harm your physical |
| | and mental health. |
| SHAM AFRAID 1 | Fear is a common and unpleasant emotion |
| | caused by the belief that someone or something |
| | is dangerous, likely to cause pain, or a threat. |
| SHAM AFRAID 2 | Fear weakens our immune system and can cause |
| | cardiovascular damage, gastrointestinal problems |
| | such as ulcers and irritable bowel syndorme, |
| | accelerated aging, and decreased fertility. |
| SHAM GRIEF 1 | After a major loss, up to a third of the people most |
| | directly affected will suffer detrimental effects on |
| | their physical or mental health, or both. |
| SHAM GRIEF 2 | Chronic stress also is common during acute grief |
| | and can lead to a variety of physical and emotional |
| | issues, such as depression, trouble sleeping, |
| | feelings of anger and bitterness, anxiety, loss of |
| | appetite, and general aches and pains. |
| SHAM STRESSED 1 | 75% of adults report experiencing moderate to |
| | high levels of stress in the past month and nearly |
| | half reported that their stress has increased in |
| | the past year. |
| SHAM STRESSED 2 | Health problems can occur if the stress response |
| | goes on for too long. Chronic stress can suppress |
| | immune, digestive, sleep, and reporductive systems. |
| SHAM DEPRESSED 1 | An estimated 17.3 million adults in the United |
| | States have had at least one major depressive |
| | episode. This number represents 7.1% of all U.S. adults. |
| SHAM DEPRESSED 2 | Depression is linked to inflammation and may change |
| | the immune system. People with depression are more |
| | likely to have inflammatory conditions or autoimmune |
| | disorders. |
| SHAM FRUSTRATED 1 | Frustration is a very common emotional response to |
| | opposition. Related to anger, annoyance, and |
| | disappointment, frustration arises from the perceived |
| | resistance to the fulfillment of an individual’s goal. |
| SHAM FRUSTRATED 2 | Some of the health problems that have been linked to |
| | unmanaged anger include: headache, digestion |
| | problems, insomnia, anxiety, depression, high blood |
| | pressure, heart attack, and stroke. |
| SHAM SHAME 1 | Shame is a painful feeling of humiliation or distress |
| | caused by the consciousness of wrong or foolish |
| | behavior. |
| SHAM SHAME 2 | Repeated humiliation, mortification, or embarrassment |
| | can cause stress, which has negative consequences |
| | for your health. |
| SHAM HAPPY 1 | Happiness is that feeling that comes over you when |
| | you know life is good and you can’t help but smile. |
| | It’s the opposite of sadness. |
| SHAM HAPPY 2 | It is a sense of well-being, joy, or contentment. When |
| | people are successful, or safe, or luck, they feel |
| | happiness. |
| SHAM OPTIMISTIC 1 | Optimism is a mental attitude reflecting a belief or |
| | hope that the outcome of some specific endeavor, or |
| | outcomes in general, will be positive, favorable, |
| | and desirable. |
| SHAM OPTIMISTIC 2 | A common idiom used to illustrate optimism is a |
| | glass filled with water to the halfway point: an optimist |
| | is said to the see the glass as half full, while a pessimist |
| | sees the glass as half empty. |
| SHAM PROUD 1 | Pride is a feeling of deep pleasure or satisfaction derived |
| | from one’s own achievements, the achievements of |
| | those with whom one is closely associated, or from |
| | qualities or possessions that are widely admired. |
| SHAM PROUD 2 | It refers to a humble and content sense of attachment |
| | towards one’s own or another’s choices and actions, |
| | or toward a whole group of people, and is a product of |
| | praise, independent self-reflection, and a fulfilled |
| | feeling of belonging. |
| SHAM MOTIVATED 1 | Someone who is motivated has a cause to do something. |
| | Being motivated causes a person to want to |
| | repeat a behavior. |
| SHAM MOTIVATED 2 | An individual’s motivation may be inspired by other |
| | people or events (extrinsic motivation), or it may come |
| | from within the individual (intrinsic motivation). |
| SHAM COMPASSIONATE 1 | Compassion is a feeling or showing sympathy and |
| | concern for others. |
| SHAM COMPASSIONATE 2 | If you are compassionate, you feel other people’s pain |
| | and struggles as though they were your own. |
| SHAM LOVING 1 | Being loving means providing and showing strong |
| | affection and attraction. |
| SHAM LOVING 2 | Being loving involves a variety of feelings, emotions, and |
| | attitudes. It often means being deeply committed |
| | and connected to someone or something. |
| SHAM CALM 1 | Calmness is the mental state of peace of mind being free |
| | from agitation, excitement, or disturbance. It also refers |
| | being in a state of serenity, tranquillity, or peace. |
| SHAM CALM 2 | A calm person does not show or feel any worry, anger, or |
| | excitement. |
| SHAM OKAY 1 | Ok means satisfactory, but not exceptionally or especially |
| | good. |
| SHAM OKAY 2 | Feeling ok means you could be better, but |
| | you could be worse. |

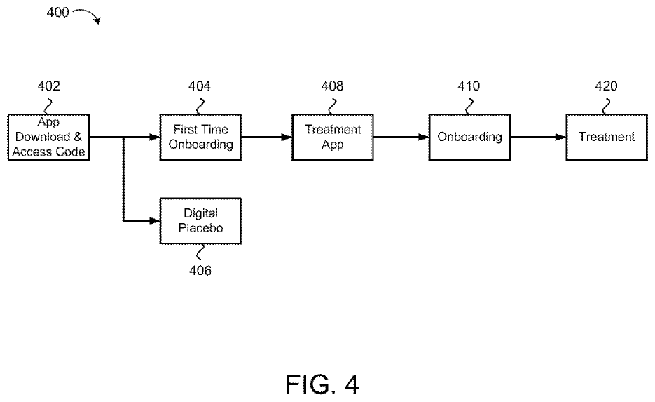

Referring to FIG. 4, a study operation 400 is generally shown in flowchart form. As set forth above, the experience of the patients in the treatment group downloading the user application 103 and receiving the access code 104 may be substantially similar to the experience of the patients in the placebo/sham group. That is, at node 402, the user application 103 may be downloaded and the patient may enter their specific access code 104. Based on the access code 104, the digital therapeutic placebo/sham application 120 determines whether the patient belongs in the treatment group or the placebo/sham group. If the digital therapeutic placebo/sham application 120 determines that the patient belongs in the treatment group, then at node 404, the authentic digital therapeutic executes the first time onboarding, including username setup, password setup, etc. If the digital therapeutic placebo/sham application 120 determines that the patient belongs in the placebo/sham group, then at node 406, the digital therapeutic placebo/sham application 120 executes the application operation 300 as described above and shown in FIGS. 3A-3C. After determining that the patient corresponds to the treatment group, the authentic digital therapeutic grants the patient access to the treatment version of the user application 103 at node 408. The authentic digital therapeutic then executes an onboarding process at node 410 to prepare the patient for the treatment. Then, at node 420, the authentic digital therapeutic executes a treatment for the patient.

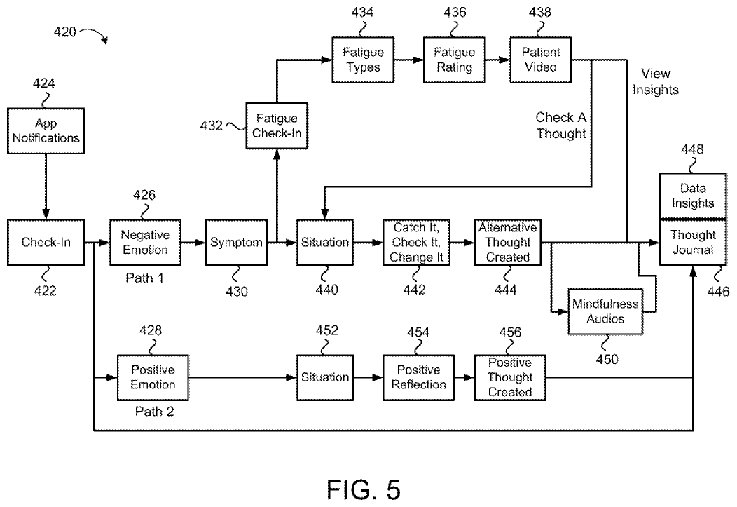

The treatment for the patient may be tailored to the specific disease or disorder that the patient is experiencing or suffering from, and, in some implementations, the treatment includes at least cognitive behavioral therapy content. By way of example, and with reference to FIG. 5, the treatment 420 is shown and described with respect to the treatment of depressive symptoms and disorders associated with multiple sclerosis. At node 422, the patient may check in to the authentic digital therapeutic via the user application 103. In some implementations, the patient may check in via one or more app notifications 424 displayed on the display 116. At the check in, the authentic digital therapeutic may receive an indication from the patient of a positive emotion (thus, leading to the first path and node 426) or a negative emotion (thus, leading to the second path and node 428). In the first path, the authentic digital therapeutic may receive from the patient an identification of one or more symptoms at node 430. As an example, if the identified symptom is fatigue, the authentic digital therapeutic presents a fatigue check-in at node 432. The authentic digital therapeutic receives an indication from the patient of what type of fatigue it is at node 434, how severe the fatigue is at node 436, and then the authentic digital therapeutic presents a patient video at node 438 and returns to path 1 at node 440.

At node 440, the authentic digital therapeutic receives a situation from the patient, and then displays a catch it, check it, change it module at node 442. After the authentic digital therapeutic instructs the patient to create an alternative thought at node 444, the authentic digital therapeutic presents a thought journal at node 446, which can be used for data insights 448. In some implementations, the authentic digital therapeutic may present mindfulness audios 450 to the patient.

In path 2, if the authentic digital therapeutic receives an indication that the patient is experiencing a positive emotion, the authentic digital therapeutic then receives a situation from the patient at node 452 and a positive reflection at node 454. After the authentic digital therapeutic instructs the patient to create positive thought at node 456, the authentic digital therapeutic presents the thought journal at node 446, which can be used for data insights 448.

In some implementations, the digital therapeutic placebo/sham application 120 may serve psycho-educational content about emotions instead of clinical content. In some implementations, a user may be presented with the same sham content if the user inputs the same conversational scenarios and/or responses.

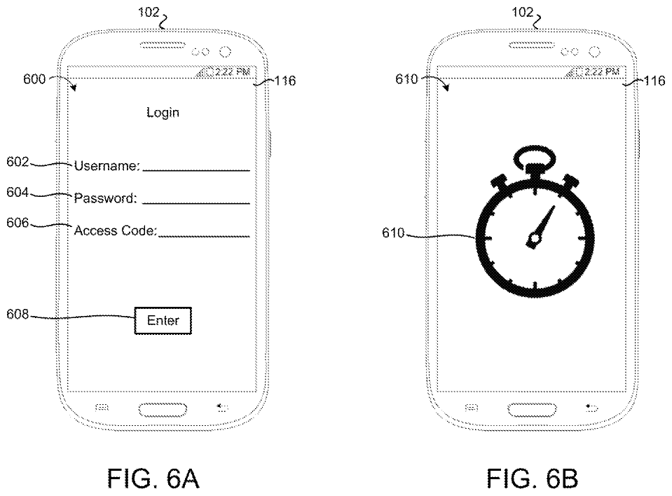

Referring to FIG. 6A, a login graphical user interface (GUI) 600 is generally shown on the display 116 of the user device 102. The login GUI 600 may include a username GUI element 602, a password GUI element 604, and an access code GUI element 606. The user 101 may interact with the username GUI element 602, the password GUI element 604, and the access code GUI element 606 to enter the user's username, password, and access code 104, respectively. The login GUI 600 may be generated and displayed by the digital therapeutic placebo/sham application 120 via the user application 103. The login GUI 600 may include a selectable enter GUI button 608 or other similar selectable GUI element.

Once the user enters the information above and selects the enter GUI 608, the digital therapeutic placebo/sham application 120 may generate and display a placebo/sham GUI 610, as shown in FIG. 6B. As set forth above, the placebo/sham GUI 610 may include a timer GUI element 612. In other implementations, the placebo/sham GUI 610 may include a blank screen, sham content, a simulated conversation for delivering sham content, etc.

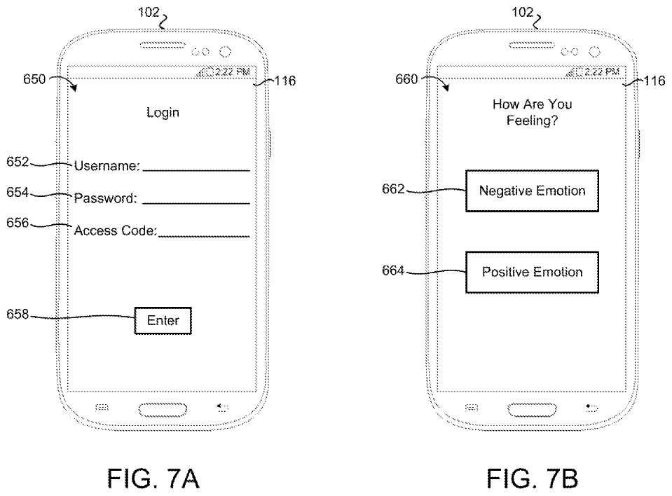

Referring to FIG. 7A, a login GUI 650 is generally shown on the display 116 of the user device 102. The login GUI 650 is the same appearance as the login GUI 600 described above, such that the user 101 is unaware as to whether they are being delivered an authentic digital therapeutic or a placebo/sham digital therapeutic. The login GUI 650 may include a username GUI element 652, a password GUI element 604, and an access code GUI element 606. The user 101 may interact with the username GUI element 652, the password GUI element 654, and the access code GUI element 656 to enter the user's username, password, and access code 104, respectively. The login GUI 650 may be generated and displayed by an authentic digital therapeutic application via the user application 103. The login GUI 650 may include a selectable enter GUI button 658 or other similar selectable GUI element.

Once the user enters the information above and selects the enter GUI button 658, the authentic digital therapeutic application may generate and display a digital therapeutic GUI 660, as shown in FIG. 7B. As set forth above, using a digital therapeutic for treating symptoms associated with multiple sclerosis, as an example, the digital therapeutic GUI 660 may include a selectable negative emotion GUI element 662 and a selectable positive emotion GUI element 664. For example, the user 101 may select the negative emotion GUI element 662 to start down the first path as described above and shown in FIG. 5. For example, the user 101 may select the positive emotion GUI element 664 to start down the second path as described above and shown in FIG. 5. In other implementations, the digital therapeutic GUI 660 may include any suitable cognitive behavioral therapy content, such as that described above by example only.

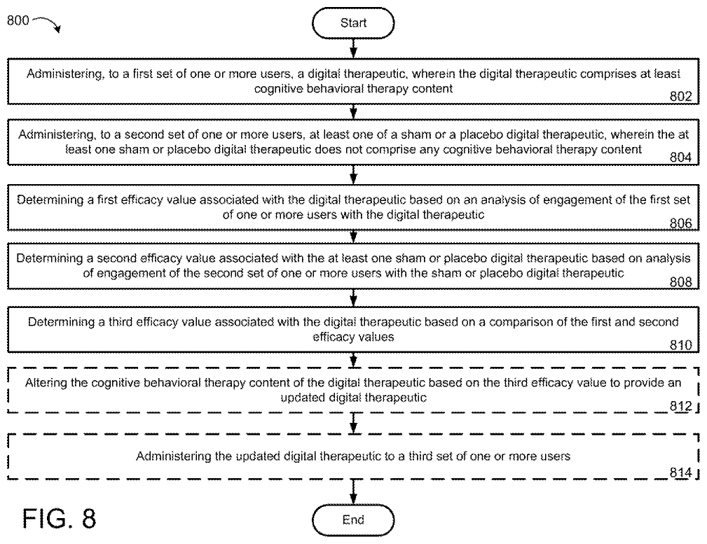

Referring to FIG. 8, a method of treatment 800 is generally shown in flowchart form. At step 802, the method 800 includes administering, to a first set of one or more users, a digital therapeutic, wherein the digital therapeutic comprises at least cognitive behavioral therapy content. For example, the first set of one or more users may include the treatment group that receive a digital therapeutic that comprises cognitive behavioral therapy content, such as the treatment 420 in the case of users diagnosed with multiple sclerosis.

At step 804, the method 800 includes administering, to a second set of one or more users, at least one of a sham or a placebo digital therapeutic, wherein the at least one sham or placebo digital therapeutic does not comprise any cognitive behavioral therapy content. For example, the second set of one or more users may include the placebo/sham group. In some implementations, the placebo/sham group may be administered the placebo digital therapeutic where no therapeutic content is delivered. For example, such placebo digital therapeutic content may include a timer or blank screen. In some implementations, the placebo/sham group may be administered the sham digital therapeutic where sham content is delivered, such as the content described in Table 1 and shown in FIG. 3C.

At step 806, the method 800 includes determining a first efficacy value associated with the digital therapeutic based on an analysis of engagement of the first set of one or more users with the digital therapeutic. For example, the digital therapeutic placebo/sham application 120 may monitor and analyze the extent, duration, frequency, etc., of the treatment group's interaction with the user application 103 to determine the first efficacy value. In some implementations, the first efficacy value is a numerical value or score, a percentage, or any other suitable type of value. In some implementations, the first efficacy value may be determined by comparing the effect of the actual or authentic digital therapeutic on a user across one or more parameters (e.g., efficacy, safety, patient adherence to a prescription regimen, mitigation of side effects, etc.) against the effect of a drug on the user across one or more of the same parameters.

At step 808, the method 800 includes determining a second efficacy value associated with the at least one sham or placebo digital therapeutic based on analysis of engagement of the second set of one or more users with the sham or placebo digital therapeutic. For example, the digital therapeutic placebo/sham application 120 may monitor and analyze the extent, duration, frequency, etc., of the placebo/sham group's interaction with the user application 103 to determine the second efficacy value. In some implementations, the second efficacy value is a numerical value or score, a percentage, or any other suitable type of value.

At step 810, the method 800 includes determining a third efficacy value associated with the digital therapeutic based on a comparison of the first and second efficacy values. For example, the digital therapeutic placebo/sham application 120 may compare the first efficacy value to the second efficacy value to determine the third efficacy value. In some implementations, the third efficacy value is a numerical value or score, a percentage, or any other suitable type of value. The third efficacy value may provide feedback regarding the efficacy of the digital therapeutic treatment compared to the placebo/sham digital therapeutic, which may function as a control.

In some implementations, the method 800 may include step 812, which includes altering the cognitive behavioral therapy content of the digital therapeutic based on the third efficacy value to provide an updated digital therapeutic. For example, the digital therapeutic placebo/sham application 120 may alter the cognitive behavioral therapy content (e.g., textual, audio, and/or visual content) that is delivered to the treatment group based on the third efficacy value, i.e., the feedback received from the comparison between the efficacy of the digital therapeutic to the efficacy of the sham or placebo digital therapeutic.

In some implementations, the method 800 may include step 814, which includes administering the updated digital therapeutic to a third set of one or more users. The third set of one or more users may include some or all of either the first or second sets of users. For example, the updated digital therapeutic may be administered to the same users that formed the basis for the third efficacy value (i.e., the determination of the efficacy of the digital therapeutic), or the updated digital therapeutic may be administered to a set of users that were not involved in the original efficacy analysis.

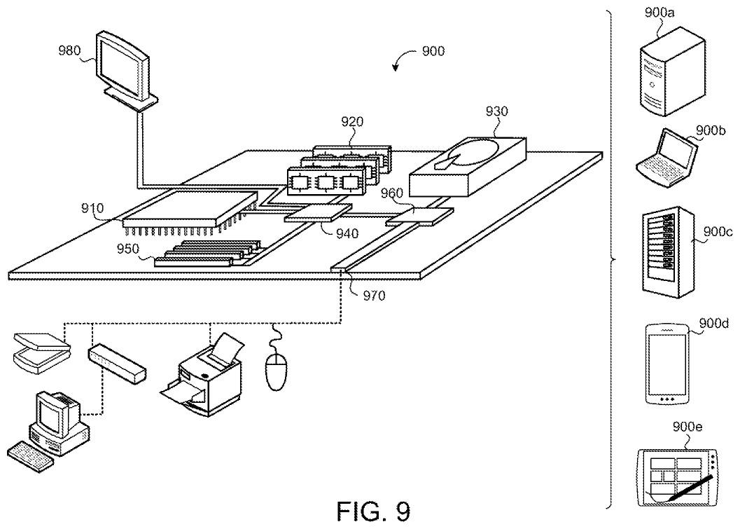

FIG. 9 is a schematic view of an example electronic device 900 (e.g., a computing device) that may be used to implement the systems and methods described in this document. The electronic device 900 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The electronic device 900 includes a processor 910, memory 920, a storage device 930, a high-speed interface/controller 940 connecting to the memory 920 and high-speed expansion ports 950, and a low speed interface/controller 960 connecting to a low speed bus 970 and a storage device 930. Each of the components 910, 920, 930, 940, 950, and 960, is interconnected using various busses, and may be mounted on a common motherboard or in other manners as appropriate. The processor 910 can process instructions for execution within the electronic device 900, including instructions stored in the memory 920 or on the storage device 930 to display graphical information for a graphical user interface (GUI) on an external input/output device, such as display 980 coupled to high speed interface 940. In other implementations, multiple processors and/or multiple buses may be used, as appropriate, along with multiple memories and types of memory. Also, multiple electronic device 900 may be connected, with each device providing portions of the necessary operations (e.g., as a server bank, a group of blade servers, or a multi-processor system).

The memory 920 stores information non-transitorily within the electronic device 900. The memory 920 may be a computer-readable medium, a volatile memory unit(s), or non-volatile memory unit(s). The non-transitory memory 920 may be physical devices used to store programs (e.g., sequences of instructions) or data (e.g., program state information) on a temporary or permanent basis for use by the electronic device 900. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 930 is capable of providing mass storage for the electronic device 900. In some implementations, the storage device 930 is a computer-readable medium. In various different implementations, the storage device 930 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 920, the storage device 930, or memory on processor 910.

The high speed controller 940 manages bandwidth-intensive operations for the electronic device 900, while the low speed controller 960 manages lower bandwidth-intensive operations. Such allocation of duties is exemplary only. In some implementations, the high-speed controller 940 is coupled to the memory 920, the display 980 (e.g., through a graphics processor or accelerator), and to the high-speed expansion ports 950, which may accept various expansion cards (not shown).

The electronic device 900 may be implemented in a number of different forms, as shown in FIG. 9. For example, it may be implemented as a standard server 900a or multiple times in a group of such servers 900a, as a laptop computer 900b, as part of a rack server system 900c, as a smartphone 900d, and/or as a tablet computer 900e.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

As used herein, the term “module” may refer to hardware, software, firmware, or any combination thereof. The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. A system for treating a disease or disorder, the system comprising:
data processing hardware; and
memory hardware in communication with the data processing hardware, the memory hardware storing instructions that when executed on the data processing hardware cause the data processing hardware to perform operations comprising:
administering, to a first set of one or more users, a digital therapeutic, wherein the digital therapeutic comprises at least cognitive behavioral therapy content;
administering, to a second set of one or more users, at least one of a sham or a placebo digital therapeutic, wherein the at least one sham or placebo digital therapeutic does not comprise any cognitive behavioral therapy content;
determining a first efficacy value associated with the digital therapeutic based on an analysis of engagement of the first set of one or more users with the digital therapeutic;
determining a second efficacy value associated with the at least one sham or placebo digital therapeutic based on analysis of engagement of the second set of one or more users with the sham or placebo digital therapeutic;
determining a third efficacy value associated with the digital therapeutic based on a comparison of the first and second efficacy values;
altering the cognitive behavioral therapy content of the digital therapeutic based on the third efficacy value to provide an updated digital therapeutic; and
administering the updated digital therapeutic to a third set of one or more users.

2. The system of claim 1, wherein the third set of one or more users may include some or all of either the first or second sets of users.

3. The system of claim 1, wherein administering the sham digital therapeutic comprises:
delivering one or more statements corresponding to general information related to at least one of an emotion, a disease, a disorder, or a condition associated with a user of the second set of one or more users.

4. The system of claim 3, wherein the one or more statements are delivered in response to the data processing hardware receiving an indication from a user of the second set of one or more users of an emotion, a disease, a disorder, or a condition that is associated with the user of the second set of one or more users.

5. The system of claim 1, wherein administering the sham digital therapeutic comprises:
generating, for display on a display device, textual content, wherein the textual content is configured to simulate a conversation with a user of the second set of one or more users.

6. The system of claim 5, wherein the textual content includes at least one of a greeting message, a prompt, a response, or an indication of the termination of the simulated conversation.

7. The system of claim 1, wherein administering the placebo digital therapeutic comprises:
generating, for display on a display device, graphical content, wherein the graphical content comprises a timer.

8. The system of claim 1, wherein administering the placebo digital therapeutic comprises:
generating, for display on a display device, a blank screen.

9. The system of claim 1, wherein administering the sham digital therapeutic comprises delivering sham content.

10. The system of claim 1, wherein administering the placebo digital therapeutic comprises delivering no content.

11. A method of treatment comprising:
administering, via data processing hardware, to a first set of one or more users, a digital therapeutic, wherein the digital therapeutic comprises at least cognitive behavioral therapy content;
administering, via the data processing hardware, to a second set of one or more users, at least one of a sham or a placebo digital therapeutic, wherein the at least one sham or placebo digital therapeutic does not comprise any cognitive behavioral therapy content;
determining, via the data processing hardware, a first efficacy value associated with the digital therapeutic based on an analysis of engagement of the first set of one or more users with the digital therapeutic;
determining, via the data processing hardware, a second efficacy value associated with the at least one sham or placebo digital therapeutic based on analysis of engagement of the second set of one or more users with the sham or placebo digital therapeutic;
determining, via the data processing hardware, a third efficacy value associated with the digital therapeutic based on a comparison of the first and second efficacy values;
altering, via the data processing hardware, the cognitive behavioral therapy content of the digital therapeutic based on the third efficacy value to provide an updated digital therapeutic; and
administering, via the data processing hardware, the updated digital therapeutic to a third set of one or more users.

12. The method of claim 11, wherein the third set of one or more users may include some or all of either the first or second sets of users.

13. The method of claim 11, wherein administering the sham digital therapeutic comprises:
delivering, via the data processing hardware, one or more statements corresponding to general information related to at least one of an emotion, a disease, a disorder, or a condition associated with a user of the second set of one or more users.

14. The method of claim 13, wherein the one or more statements are delivered in response to the data processing hardware receiving an indication from a user of the second set of one or more users of an emotion, a disease, a disorder, or a condition that is associated with the user of the second set of one or more users.

15. The method of claim 11, wherein administering the sham digital therapeutic comprises:
generating, via the data processing hardware, for display on a display device, textual content, wherein the textual content is configured to simulate a conversation with a user of the second set of one or more users.

16. The method of claim 15, wherein the textual content includes at least one of a greeting message, a prompt, a response, or an indication of the termination of the simulated conversation.

17. The method of claim 11, wherein administering the placebo digital therapeutic comprises:
generating, via the data processing hardware, for display on a display device, graphical content, wherein the graphical content comprises a timer.

18. The method of claim 11, wherein administering the placebo digital therapeutic comprises:
generating, via the data processing hardware, for display on a display device, a blank screen.

19. The method of claim 11, wherein administering the sham digital therapeutic comprises delivering, via the data processing hardware, sham content.

20. The method of claim 11, wherein administering the placebo digital therapeutic comprises delivering, via the data processing hardware, no content.
